(19) United States
(12) Patent Application Publication
(10) Pub. No.: US 2019/0011349 A1
(43) Pub. Date: Jan. 10, 2019

(54) LABEL-FREE CHARACTERIZATION OF PARTICLES SUSPENDED IN A FLUID

(71) Applicant: THE BOARD OF TRUSTEES OF THE UNIVERSITY OF ILLINOIS, Urbana, IL (US)

(72) Inventors: Rashid BASHIR, Champaign, IL (US); Bobby REDDY, Jr., Urbana, IL (US); Tanmay GHONGE, Urbana, IL (US); Umer HASSAN, Urbana, IL (US); Gary DURACK, Urbana, IL (US); Gregory DAMHORST, Elgin, IL (US)

(73) Assignee: THE BOARD OF TRUSTEES OF THE UNIVERSITY OF ILLINOIS, Urbana, IL (US)

(21) Appl. No.: 16/068,945

(22) PCT Filed: Jan. 12, 2017
(86) PCT No.: PCT/US2017/013155

§ 371 Date: Jul. 10, 2018

Related U.S. Application Data
(60) Provisional application No. 62/277,736, filed on Jan. 12, 2016.

(51) Int. Cl. G01N 15/10; G01N 33/487; C12M 1/34

(52) U.S. Cl. CPC G01N 15/1031; G01N 33/48721; G01N 2015/1486; G01N 2015/1006; C12M 41/36

(57) ABSTRACT

Provided are methods and systems that characterize a property of a particle suspended in a fluid sample in a label-free manner. Detection elements are provided fluidically adjacent upstream and downstream from a modulation element. Fluid sample containing particles flows across a first detection element and a first particle parameter detected for each particle that passes the first detection element or a first aggregate particle parameter for a plurality of particles that pass the first detection element. The particles flow from the first detection element to a first modulation element, wherein the first modulation element effects a change in a property of the particles flowing past the first modulation element. A second detection element then detects the particle parameter again or a second aggregate particle parameter for a plurality of particles that pass the second detection element. Comparing the first and second particle or aggregate parameters thereby characterizes the particle property.

### CROSS-REFERENCE TO RELATED APPLICATIONS

This application claims the benefit of U.S. Provisional Patent Application No. 62/277,736 filed Jan. 12, 2016, which is hereby incorporated by reference to the extent not inconsistent herewith.

### STATEMENT REGARDING FEDERALLY SPONSORED RESEARCH OR DEVELOPMENT

Not applicable.

### BACKGROUND OF INVENTION

Provided are methods and systems for characterizing particle properties for particles suspended in a fluid in a manner that is label free and electronic based. The methods and systems are particularly useful for detecting and quantifying various biomarkers from blood.

Many conventional assays for detecting biomarkers require labels and/or excitation light sources, including excitation lasers, to detect cell surface proteins or plasma biomarkers. Such assays suffer from a number of fundamental disadvantages. For example, the labeling oftentimes results in particle destruction so that the particles can only be analyzed once for a single biomarker. This makes such assays fundamentally incompatible with multiplexing where a plurality of biomarkers is analyzed with repeated tests. Furthermore, the conventional assays require expensive and complex optical components along with attendant need to store data-intensive image files, including for subsequent analysis. These requirements make the ability to incorporate such assays in a hand-held device, at best, impractical. Accordingly, there is a need for a device where the output from a sensor of the device is modulated by an intrinsic property of the particles suspended in the fluid.

Microcytometers have been proposed and described, including a “device to electrically count blood cell populations using an AC impedance interrogation technique in a microfabricated cytometer (microcytometer).” Watkins et al. Lab on a Chip 9 (3177) (2009) (abstract). Differential counting is described in Watkins et al. Lab on a Chip 11 (1437) (2011) (“T cell counts are found by obtaining the difference between the number of leukocytes before and after depleting CD4+ T cells with immobilized antibodies in a capture chamber.” Abstract), and Science Translational Medicine 5 (214) (2013). Those devices and systems are further discussed in U.S. Pub. No. 2013/0295588.

There continues to be a need, however, for reliable and robust handheld devices so that healthcare professions can perform complete blood diagnostics at the point of patient care. Such a device can facilitate early-stage disease diagnosis before serious and potentially debilitating symptoms appear. Furthermore, a point of care testing provides rapid diagnostic tests to be performed at a patient bedside, or even in the field where a medical facility is not readily available. Immediate relevant data specific to a patient is obtained that can, in turn, be immediately interpreted by the person holding the hand-held device and is, therefore, amenable to interpretation by a wide range of health professionals and is not confined to a medical doctor, including a nurse or trained technician.

### SUMMARY OF THE INVENTION

Provided herein is a method, and related devices that incorporate the method, having a measured output from sensor-type components of the device that is modulated by an intrinsic property of the particles suspended in the fluid. The system and methods are uniquely configured using modular detection elements and modulation elements arranged in various patterns to characterize a particle property of a particle suspended in a flowing fluid solution. In this manner, the modularity and flexibility associated with the pattern of detection and modulation elements ensures the systems and methods are compatible for a range of applications, each application having specific fluid samples, particles and biomarker(s) that are desirably characterized.

As particles flow past a detection element, including in a single file, a set of electrical properties and a time stamp may be recorded, including on an individual (“particle-by-particle”) basis or on a population-level basis. A modulation zone is fluidically connected to the detection element, so that after the particles have flown past the detection element they are introduced to the modulation element. “Fluidically connected” refers to a combination of components that are connected so that a fluid is capable of flowing between the components without adversely impacting the functionality of each component. Accordingly, the modulation zone is configured to be capable of interacting with desired particles in a manner that is conducive for subsequent characterization. This interaction is used herein broadly, and can refer to a particle parameter, such as a change in particle velocity, particle capture, or modification to the particle that can be subsequently determined, including by any of the detection elements. In this manner, particles exiting the modulation element are fluidically introduced to a downstream detection element where a second set of electrical properties and time stamps are measured and recorded, either on a particle-by-particle basis or a population level basis. The electrical properties and time stamp from the upstream and downstream detection elements are compared so that information about the particle and/or biomarker is obtained. The electrical properties may be associated with one or more of a particle electrical property, mechanical property or a magnetic property.

In this manner, a single unified platform is possible for all relevant biomarkers, including cell counts, surface protein expression or concentration, fluid biomarkers, including plasma proteins, nucleic acids and small molecules. The approaches provided herein are further advantageous in that they are readily scalable for multiplexing of many biomarkers by use of spatially distinct modulation zones within the same device. This is a fundamental improvement over conventional optically-based techniques. Accordingly, a need for optical components and labeling is eliminated, thereby increasing the feasibility of cost-efficient point of care devices.

The methods and systems provide a number of benefits, including adaptability and versatility in that they can be readily tailored to wide number of applications, scalability, and cost effectiveness. Additional benefits include a characterization of particles, such as biomarkers from blood, which is immediately available for interpretation. Minimal sample processing, including label-free testing, results in decreased cost and effort, which directly impacts frequency and availability of patient testing. Furthermore, the methods and systems may be utilized as a cost-effective method to produce a patient biomarker profile, including for a plurality of biomarkers. This can provide an effective patient management platform, including for diagnosis and prevention of disease, particularly for diseases having a particular biomarker profile.

The applications compatible with the systems and methods provided herein are varied. Specific examples include counting the number of functionalized beads on which a biomarker analyte has been captured to measure proteins or DNA, including the number of captured functionalized beads. Another example is using a measured transit time of cells with surface antigens across a channel coated with complementary antibodies to measure expression of specific protein receptors on the cell surface.

The modular nature of the modulation and detection elements provides for flexibility and allows for use of these elements in series, in parallel, or combinations thereof. The parameters modulated by the modulation elements may include, but do not need to be limited to, capture of particles, increased transit time of particles, or modification of particles. A modulation element is paired to upstream and downstream detection elements that can both record a particle parameter on a single particle-by-particle basis such as time stamp, size and dielectric properties. By measuring the change induced in the particle and/or its traversal time through the modulation element, this overall system can measure a wide variety of particle properties, including surface expression of molecules on a particle, including an artificial bead or a biological cell. Because the system, for each modulation element and adjacent detection elements, can measure surface expression, arranging a plurality of modulation elements, each targeting a different surface molecule, the system can be used as a multiplexed platform for cell counting, cell surface proteins, and plasma or other fluid biomarkers, including but not limited to proteins and nucleic acids. The scalability of the system is evident by the fact that many modulation zones may be used on a single chip. This is extremely attractive when considering the conventional systems that use one color of light and one label per analyte.

Provided herein are various label-free methods for characterizing a property of a particle suspended in a fluid sample. For example, the method may comprise the steps of: flowing a fluid sample containing particles across a first detection element, wherein the particles flow in substantially single file across the first detection element; detecting a first particle parameter for each particle that passes the first detection element to obtain a first aggregate particle parameter for a plurality of particles that pass the first detection element; flowing the particles from the first detection element to a first modulation element, wherein the first modulation element effects a change in a property of the particles or a particle flow parameter of the particles flowing past the first modulation element; flowing the particles from the first modulation element across a second detection element, wherein the particles flow in substantially single file across the second detection element; detecting a second aggregate particle parameter for a plurality of particles that pass the second detection element; comparing the first aggregate particle parameter with the second aggregate particle parameter; thereby characterizing the particle property. The methods and systems are compatible with a wide range of particle property characterization. Examples include one or more of: biomolecule presence on a surface of the particle; biomolecule surface concentration on a surface of the particle; biomolecule presence in the fluid sample; and biomolecule concentration in the fluid sample.

As discussed herein, the methods are particularly useful for multiplex characterization of a plurality of particle properties and/or a plurality of particle populations. For example, the method may further comprise the steps of: repeating the flowing steps for one or more additional detection elements and one or more modulation elements to obtain one or more additional aggregate particle parameters and particle properties, thereby providing a multiplex characterization for a plurality of particle properties.

The additional detection and modulation elements are positioned as desired depending on the application of interest, such as provided in a parallel configuration, a series configuration, or a combination of parallel and series configuration. For simplicity the detection elements that are fluidically up- and downstream of the modulation element may correspond to a single detection element, where the fluid flow conduit that receives fluid from the modulation element is directed back to the single detection element.

At least one particle property may provide information about a biomarker that is a receptor on a surface of the particle. The biomarker may be a naturally-occurring receptor on a biological cell membrane or a receptor that is connected to an artificial particle, such as a microsphere.

The comparing step may comprise determining one or more of: a time elapsed between the particles that pass the first detection element and the particles that pass the second detection element; or particle flux or spacing. In this manner, a measure of a particle transit time through the modulation element with an associated non-optical characterization of the particle property can be obtained.

The detection element may detect a physical property of the particle selected from the group consisting of: an electrical property, an optical property, a magnetic property, or a mechanical property; wherein a change in the detected physical property between the first and second detection element provides the particle property characterization.

The detection element may comprise an electrode to detect a change in an electrical property when a particle passes the detection element. The electrical property may itself provide useful information, including a simple confirmation about when the particle is detected. Additional information may be provided related to a property that can assist in distinguishing and/or identifying different populations. For example, different size particles may provide a different impedance, resistance, capacitance or the like detected by detection element.

The first and second detection elements may be a common detection element or they may be different and distinct detection elements.

Any of the methods and systems provided herein may have at least one detection element configured to distinguish a plurality of particle populations. For example, the plurality of particle populations may be distinguished based on an electrical property, including a change in impedance as a particle passes the detector, with a first population of particles associated with a first average impedance value and a second population of particles associated with a second average impedance value.

Depending on the application of interest, the methods and systems provided herein are compatible with a range of aggregate particle parameter types. For example, the first and/or second aggregate particle parameters may be one or more of: impedance, resistance, current, optical intensity, transit time, velocity, refractive index, viscosity, a magnetic parameter, a mechanical parameter such as stiffness, a property of a constituent of the particles including a nucleus of a biological cell.

The detection element may be further characterized as having an interrogation zone in which the first or second aggregate particle parameter is measured.

The modulation element may comprise a plurality of modulation element surface-bound targets that specifically interacts, including by binding, to a counter-analyte on a surface of the particle, wherein the interaction results in particle adherence to a surface of the modulation element or particle rolling over the surface of the modulation element; a geometry configured to assess a particle physical parameter, such as stiffness, viscosity, density, size, refractive index, charge; and/or a chemical agent to modify a particle characteristic. With respect to a receptor-ligand type interaction, the methods and systems are compatible with the receptor on either of the particle surface or a contact surface of the modulation element, with the associated ligand either on the contact surface of the modulation element or the particle (or within the fluid flowing over the contact surface), respectively. The geometry can refer to a size, shape, and/or position of, for example, a constriction, so that the physical interaction between particle and surface impacts transit time through the modulation element, dependent on particle size and/or physical characteristic such as stiffness. Chemical agent refers to a material that effects a change in the particle, such as a change resulting from a signal cascade arising from binding or a change resulting from a chemical modification.

The modulation element may comprise a plurality of surface-bound targets selected from the group consisting of: a polypeptide sequence; a polynucleotide sequence; a protein; an antibody; an antigen; and a chemical substance having activity for a biomolecule of interest.

The modulation element may generate a modulation force on the particle, the modulation force selected from the group consisting of: an antibody affinity; an optical force; a dielectrophoretic force; a lateral flow force; a microfluidic force generated by a fluidic geometry of the modulation element; a chemically-generated force.

The modulation element may provide one or more of: decrease in a velocity of the particle; adherence of the particle to a surface of the modulation element; or a modification of the particle.

The methods and systems are compatible with a range of particle types, sizes and origin. For example, the particle may be selected from the group consisting of one or more of a biological cell; a microsphere; a charged species; a protein; a polypeptide, DNA, RNA, a polynucleotide; an antibody; and an antigen. Specific examples of particles include a biological cell from a blood sample, such as a leukocyte. Exemplary particle sizes include particles that are cellular sized having an average diameter of between 5 μm and 25 μm, or even smaller sizes for applications of interest related to sub-cellular sized particles, including a charged species, protein, polypeptide, DNA, RNA, polynucleotide, antibody and antigens. Accordingly, the particle size may span into the sub-micron range, such as between 1 nm and 1 μm, or, more generally, between 1 nm and 25 μm, and any sub-ranges thereof.

The method may further comprise diluting the fluid sample to avoid simultaneous particle detection by the first detection element or the second detection element. The desired particle concentration may be calculated, based on the average fluid flow-rate and the volume of space in which it is desired to have only one particle present. In addition, on chip strategies may be used to decrease the probability of simultaneous particle detection even with high initial particle concentrations, such as with fluidic controls, including gating and flow regulation. Statistical algorithms may also be applied to account for cases where simultaneous particle detection is unavoidable.

The particle may be a biomaterial isolated from a biological sample; or a material that specifically captures a biomaterial from a biological sample, such as a microsphere configured to capture the biomaterial.

The method and systems are compatible with a plurality of distinct particle populations, with a particle parameter characterization for each of the distinct populations.

The biomolecule may be selected from the group consisting of: a cell surface receptor; plasma proteins, plasma nucleic acids, small molecules, a biomaterial released from a lysed cell; a bacteria, a virus; mRNA, DNA, miRNA, a parasite. Other biomolecules of interest may be selected depending on the application of interest. For example, components of interest in urinalysis may include: proteins, cells and cellular casts, sugars, ions, crystals, hormones (peptides or small molecules), bacteria, pH. For analysis of cerebrospinal fluid (CSF), analytes of interest are generally similar. Accordingly, more generally a biomolecule herein may refer to a component of biological fluid as well as components released from cells in biological fluid. The biomolecule may be a pathogen, including viruses, bacteria, and parasites. The biomolecule may be a nucleic acid, including DNA, RNA, mRNA, miRNA, and portions thereof. The biomolecule may be a protein, a peptide, a small molecule, or a carbohydrate. The breadth of biomolecules useful with the processes and devices described herein reflects the versatility and compatibility of the processes and devices for a range of applications.

The methods and systems have use in a varied range of applications, including one or more of: particle counting; particle sorting; surface protein expression; plasma protein level measurement; nucleic acid detection; small molecule detection; particle motility; co-expression detection of multiple biomolecules; expression of plasma proteins or nucleic acid within a biological cell; electrolyte characterization; and quality control. In an aspect, the method and system is for an application that is not simply particle counting alone, but may have particle counting with at least one other application.

The modulation element may be selected to provide an assessment of: cell activity; cell surface protein; plasma proteins; and/or plasma nucleic acids.

The method and systems may be used in a point of care device, thereby avoiding the need for laboratory detection and associated sample processing, handling and testing.

The method and systems may be used to measure cell surface antigen expression, and/or co-expression of a plurality of cell surface markers.

Any of the methods and systems may further comprise the step of generating histograms of detected particles as a function of elapsed time between detection of the first particle parameter and the second particle parameter.

The comparing step may comprise determining a difference between the first particle parameter and the second particle parameter and plotting a histogram of the difference for the particles in the fluid sample.

The method and systems may be further characterized in terms of a total multiplexing number that is the product of the total number of modulation elements and the total number of populations distinguished by the detection elements. The total multiplexing number may be greater than or equal to 6.

The method may further comprise the step of optimizing the modulation element to control a number of captured particles by the modulation element. The optimizing may comprise one or more of: selecting a shear force at the modulation element wall; incubating particles in the modulation element for an incubation time; or selecting a target element density on the modulation element wall.

The method or system may be for quantifying surface expression of biomolecules on a particle surface. For example, the quantifying may be by counting a number of particles captured by the modulation element having a surface coating of target molecules specific for the biomolecules on the particle surface. The method may also be for a particle that is a bead and the biomolecules on the bead surface correspond to a biomaterial isolated from a biological fluid that are attached directly or indirectly, to the bead surface, including by a covalent attachment to a linker moiety connected to the bead surface.

Also provided herein are systems for multiplexed detection of biomarkers on a particle surface. The system may comprise: a plurality of detection elements, wherein the detection elements are configured to detect a passing particle based on an electrical parameter associated with the particle passing the detection element; a plurality of modulation elements, wherein adjacent detection elements are separated by a modulation element, wherein each modulation element comprises a functionalized surface that is different in composition from a functionalized surface of another modulation element; a fluid conduit that fluidically connects adjacent detection and modulation elements for providing particles suspended in a fluid to the detection and modulation elements; an electronic system configured to: obtain an electrical parameter for each particle that passes each detection element; obtain an aggregate particle parameter from a plurality of particles that passes the detection element, wherein each detection element has a unique aggregate particle parameter; detect a plurality of biomarkers by comparing the aggregate particle parameters from adjacent detection elements separated by one of the modulation elements; a microfluidic pump for forcing the particles suspended in the fluid through the plurality of detection elements and the plurality of modulation elements.

The fluid conduit has at least a portion with a cross-sectional area selected to facilitate single-file flow of particles over each detection element and each modulation element. For example, the conduit may have a dimension that is between 1 D and 10 D, or between 1.5 D and 10 D, wherein D is an average particle diameter and flow is laminar.

The particles may interact with a surface of the modulation element, thereby facilitating various interactions, such as an adherence interaction (e.g., long-term interaction), a rolling interaction (e.g., short or temporary and repeated interactions that slows the particle), or a free-flow velocity that is not substantially decreased by the functionalized surface (e.g., non-interacting).

The detection element may comprise an electrode.

The functionalized surface of the modulation element may comprise a target molecule specific for a biomarker on the particle surface, including to provide a receptor-ligand interaction.

The detection and modulation elements may be arranged in a series configuration, a parallel configuration, or both a series and a parallel configuration.

The detection elements may be re-useable and the modulation elements may be replaceable, including modulation elements that are positioned within a removable cartridge in a point-of-care device.

Aspects of the invention are provided as in the following numbered embodiments:

1. A label-free method for characterizing a property of a particle suspended in a fluid sample, the method comprising the steps of: flowing a fluid sample containing particles across a first detection element, wherein the particles flow in substantially single file across the first detection element; detecting with the first detection element a particle parameter for at least a portion of the particles that pass the first detection element; flowing the particles from the first detection element to a first modulation element, wherein the first modulation element effects a change in the first particle parameter of the particles flowing past the first modulation element; flowing the particles from the first modulation element across a second detection element, wherein the particles flow in substantially single file across the second detection element; detecting with the second detection element the particle parameter for the at least a portion of the particles that pass the second detection element, wherein the particle parameter detected by the second detection element has a value that is different than a value of the particle parameter detected by the first detection element; comparing the particle parameter detected by the first detector with the particle parameter detected by the second detector; thereby characterizing the particle property; wherein the particle property is selected from the group consisting of: biomolecule presence on a surface of the particle; biomolecule surface concentration on a surface of the particle; biomolecule presence in the fluid sample; and biomolecule concentration in the fluid sample.

2. A label-free method for characterizing a property of a particle suspended in a fluid sample, the method comprising the steps of: flowing a fluid sample containing particles across a first detection element, wherein the particles flow in substantially single file across the first detection element; detecting a first particle parameter for each particle that passes the first detection element to obtain a first aggregate particle parameter for a plurality of particles that pass the first detection element; flowing the particles from the first detection element to a first modulation element, wherein the first modulation element effects a change in a property of the particles of the particles flowing past the first modulation element; flowing the particles from the first modulation element across a second detection element, wherein the particles flow in substantially single file across the second detection element; detecting a second aggregate particle parameter for each particle that passes the second detection element to obtain a second aggregate particle parameter for a plurality of particles that pass the second detection element; comparing the first aggregate particle parameter with the second aggregate particle parameter; thereby characterizing the particle property; wherein the particle property is selected from the group consisting of: biomolecule presence on a surface of the particle; biomolecule surface concentration on a surface of the particle; biomolecule presence in the fluid sample; and biomolecule concentration in the fluid sample.

3. The method of claim 1 or 2, further comprising the steps of: repeating the flowing steps for one or more additional detection elements and one or more modulation elements to obtain one or more additional particle parameters or aggregate particle parameters and particle properties, thereby providing a multiplex characterization for a plurality of particle properties.

4. The method of claim 3, wherein the additional detection and modulation elements are provided in a parallel configuration, a series configuration, or a combination of parallel and series configuration.

5. The method of any of claims 1-4, wherein at least one particle property provides information about a biomarker that is a receptor on a surface of the particle.

6. The method of any of claims 1-5, wherein the comparing step comprises determining: a time elapsed between the particles that pass the first detection element and the particles that pass the second detection element; or particle flux or spacing; thereby obtaining a measure of a particle transit time through the modulation element and non-optically characterizing the particle property.

7. The method of claim 1, wherein the detection element detects a physical property of the particle selected from the group consisting of: an electrical property, a magnetic property, and a mechanical property; wherein a change in the detected physical property between the first and second detection element provides the particle property characterization.

8. The method of claim 2, wherein the detection element detects a physical property of the particle selected from the group consisting of: a mechanical property; and a magnetic property; wherein a change in the detected physical property between the first and second detection element provides the particle property characterization.

9. The method of claim 1, wherein the detection element comprises an electrode to detect a change in an electrical property when a particle passes the detection element.

10. The method of any of claims 1-9, wherein the first and second detection elements are a common detection element.

11. The method of any of claims 1-10, wherein the first and second detection elements are different detection elements.

12. The method of any of claims 1-11, wherein at least one detection element is configured to distinguish a plurality of particle populations.

13. The method of claim 12, wherein the plurality of particle populations are distinguished based on an electrical property, including a change in impedance as a particle passes the detector, with a first population of particles associated with a first average impedance value and a second population of particles associated with a second average impedance value.

14. The method of claim 2, wherein the first or second aggregate particle parameter is selected from the group consisting of: impedance, resistance, current, transit time, velocity, refractive index, viscosity, a magnetic parameter, a mechanical parameter such as stiffness, and a property of a constituent of the particles including a nucleus of a biological cell.

15. The method of any of claims 1-14, wherein the detection element has an interrogation zone in which the particle parameter or the first or second aggregate particle parameter is measured.

16. The method of any of claim 1-15, wherein the modulation element comprises: a plurality of modulation element surface-bound targets that specifically bind to a counter-analyte on a surface of the particle, wherein the binding results in particle adherence to a surface of the modulation element or particle rolling over the surface of the modulation element; a geometry configured to assess a particle physical parameter, such as stiffness, viscosity, density, size, refractive index, charge; and/or a chemical agent to modify a particle characteristic.

17. The method of any of claims 1-16, wherein the modulation element comprises a plurality of surface-bound targets selected from the group consisting of: a polypeptide sequence; a polynucleotide sequence; a protein; an antibody; an antigen; and a chemical substance having activity for a biomolecule of interest.

18. The method of any of claims 1-17, wherein the modulation element generates a modulation force on the particle, the modulation force selected from the group consisting of: an antibody affinity; an optical force; a dielectrophoretic force; a lateral flow force; a microfluidic force generated by a fluidic geometry of the modulation element; and a chemically-generated force.

19. The method of any of claims 1-18, wherein the modulation element provides one or more of: decrease in a velocity of the particle; adherence of the particle to a surface of the modulation element; or a modification of the particle.

20. The method of any claims 1-19, wherein the particle is selected from the group consisting of one or more of a biological cell; a microsphere; a charged species; a protein; a polypeptide, DNA, RNA, a polynucleotide; an antibody; and an antigen.

21. The method of claim 20, wherein the particle is a biological cell from a blood sample.

22. The method of claim 21, wherein the particle is a leukocyte.

23. The method of claim 1 or 2, wherein the particle has an average diameter of between 5 μm and 25 μm.

24. The method of any of claims 1-23, further comprising diluting the fluid sample to avoid simultaneous particle detection by the first detection element or the second detection element.

25. The method of any of claims 1-24, wherein the particle comprises: a biomaterial isolated from a biological sample; or a material that specifically captures a biomaterial from a biological sample.

26. The method of any claims 1-25, wherein there is a plurality of distinct particle populations, and the method characterizes a particle parameter for each of the distinct populations.

27. The method of any of claims 1-26, wherein the particle property biomolecule is selected from the group consisting of: a cell surface receptor; plasma proteins, plasma nucleic acids, small molecules, a biomaterial released from a lysed cell; a bacteria, a virus; mRNA, and DNA.

28. The method of any of claims 1-27 used in an application selected from the group consisting of one or more of: particle counting; particle sorting; surface protein expression; plasma protein level measurement; nucleic acid detection; small molecule detection; particle motility; co-expression detection of multiple biomolecules; expression of plasma proteins or nucleic acid within a biological cell; electrolyte characterization; and quality control.

29. The method of any of claims 1-28, wherein the modulation element is selected to provide an assessment of: cell activity; cell surface protein; plasma proteins; and/or plasma nucleic acids.

30. The method of any of claims 1-29 used in a point of care device.

31. The method of claim 1 or 2, used to measure cell surface antigen expression.

32. The method of claim 3, to measure co-expression of a plurality of cell surface markers.

33. The method of any of claims 1-32, further comprising the step of generating histograms of detected particles as a function of elapsed time between detection of the particle parameter with the first and second detection elements or the first and second aggregate particle parameters.

34. The method of any of claim 32-33, wherein the comparing step comprises determining a difference between the particle parameters detected by the first and second detection elements or the first aggregate particle parameter and the second aggregate particle parameter, and plotting a histogram of the difference for the particles in the fluid sample.

35. The method of any claims 1-34 that provides a total multiplexing number that is the product of the total number of modulation elements and the total number of populations distinguished by the detection elements, wherein the total multiplexing number is greater than or equal to 6.

36. The method of any of claims 1-35, further comprising the step of optimizing the modulation element to control a number of captured particles by the modulation element.

37. The method of claim 36, wherein the optimizing comprises one or more of: selecting a shear force at the modulation element wall; incubating particles in the modulation element for an incubation time; or selecting a target element density on the modulation element wall.

38. The method of any claims 1-37, for quantifying surface expression of biomolecules on a particle surface.

39. The method of claim 38, wherein the quantifying is by counting a number of particles captured by the modulation element having a surface coating of target molecules specific for the biomolecules on the particle surface.

40. The method of claim 38, wherein the particle is a bead and the biomolecules on the bead surface correspond to a biomaterial isolated from a biological fluid.

41. A system for multiplexed detection of biomarkers on a particle surface comprising: a plurality of detection elements, wherein the detection elements are configured to detect a passing particle based on an electrical parameter associated with the particle passing the detection element; a plurality of modulation elements, wherein adjacent detection elements are separated by a modulation element, wherein each modulation element comprises a functionalized surface that is different in composition from a functionalized surface of another modulation element; a fluid conduit that fluidically connects adjacent detection and modulation elements for providing particles suspended in a fluid to the detection and modulation elements; an electronic system configured to: obtain an electrical parameter for each particle that passes each detection element, wherein a modulation element positioned between adjacent detection elements is configured to generate a change in the obtained electrical parameter; and detect a plurality of biomarkers by comparing the obtained particle parameters from adjacent detection elements separated by one of the modulation elements; a microfluidic pump for forcing the particles suspended in the fluid through the plurality of detection elements and the plurality of modulation elements.

42. A system for multiplexed detection of biomarkers on a particle surface comprising: a plurality of detection elements, wherein the detection elements are configured to detect a passing particle based on an electrical parameter associated with the particle passing the detection element; a plurality of modulation elements, wherein adjacent detection elements are separated by a modulation element, wherein each modulation element comprises a functionalized surface that is different in composition from a functionalized surface of another modulation element; a fluid conduit that fluidically connects adjacent detection and modulation elements for providing particles suspended in a fluid to the detection and modulation elements; an electronic system configured to: obtain an electrical parameter for each particle that passes each detection element; obtain an aggregate particle parameter from a plurality of particles that passes the detection element, wherein each detection element has a unique aggregate particle parameter; detect a plurality of biomarkers by comparing the aggregate particle parameters from adjacent detection elements separated by one of the modulation elements; a microfluidic pump for forcing the particles suspended in the fluid through the plurality of detection elements and the plurality of modulation elements.

43. The system of claim 41 or 42, wherein the conduit has a cross-sectional area selected to facilitate single-file flow of particles over each detection element and each modulation element.

44. The system of any of claims 41-43, wherein the conduit has a dimension that is between 1.5 D and 10 D, wherein D is an average particle diameter and flow in the conduit is laminar.

45. The system of any of claims 41-44, wherein particles interact with a surface of the modulation element.

46. The system of claim 45, wherein the interaction is an adherence interaction, a rolling interaction, or a free-flow velocity that is not substantially decreased by the functionalized surface.

47. The system of claim 41, wherein the detection element comprises an electrode.

48. The system of any of claims 41-47, wherein the functionalized surface of the modulation element comprises a target molecule specific for a biomarker on the particle surface.

49. The system of any of claims 41-48, wherein the detection and modulation elements are arranged in a series configuration, a parallel configuration, or both a series and a parallel configuration.

50. The system of any of claims 41-49, wherein the detection elements are re-useable and the modulation elements are replaceable.

51. The system any of claims 41-50, where the modulation elements are positioned within a removable cartridge in a point-of-care device.

52. The system of claim 41, wherein the detection element detects a physical property of the particle selected from the group consisting of: an electrical property, a mechanical property; and a magnetic property.

53. The system of claim 42, wherein the detection element detects a physical property of the particle selected from the group consisting of: a mechanical property; and a magnetic property.

54. The system of any of claims 41-53, comprising three or more detection elements.

Without wishing to be bound by any particular theory, there may be discussion herein of beliefs or understandings of underlying principles relating to the devices and methods disclosed herein. It is recognized that regardless of the ultimate correctness of any mechanistic explanation or hypothesis, an embodiment of the invention can nonetheless be operative and useful.

### BRIEF DESCRIPTION OF THE DRAWINGS

FIG. 1. Modulation elements useful with the methods and systems provided herein for biological cells (top left panel) and synthetic beads of microparticles (top right panel). The term “bead based ELISA” is a short-hand characterization that refers to free analyte capture with a functionalized bead. Unlike conventional ELISA's, an enzyme is not needed in this illustrated example. The modulation element is exemplified as an antibody attached to a surface or membrane that may interact with relevant targets on cell or microparticle surface, as desired.

FIG. 2. (a) Schematic illustrating operation of a detection element having an interrogation zone. (b) An exemplary table of properties that can be assessed on a particle by particle level to illustrate the versatility of the methods and systems, including the ability to for multiplex detection and characterization. (c) Histograms illustrating data on a population level.

FIG. 3. (a) Schematic illustrating the input and output of a modulation element. Some particles may be slowed by the modulation (small circles, indicative of transient interaction and resultant decrease in average particle velocity compared to bulk fluid, also referred herein as “rolling”), some particles may be captured (rectangles, indicative of particle adherence to the surface), some particles may be modified (squares), and some may be unaffected (larger circles, whose average velocity is equivalent to bulk fluid velocity). (b)-(e) schematics illustrating various possibilities for modulation elements, including: (b) unaffected; (c) rolling; (d) capture; (e) modified, respectively.

FIG. 4. a Schematic illustrating a simple example of a detection-modulation-detection elements configuration, with the detection elements corresponding to a single detection element, where after passing through the modulation element, the particle flow is passed back over the detection element, but with a different detection parameter, as indicated by the Detection(i) and Detection(j) labels. b Top: Table showing possible measured entities on a particle by particle basis for both the first detection (i) and the second detection (j). Time stamp refers to the ability to characterize the time of travel across Modulation Element by determining elapsed time between first Detection (i) and second Detection (j). Bottom: Comparison of the properties and time stamp for the particles on an individual particle basis.

FIG. 5. a Difference in property A before and after modulation element showing very little change. b Difference in property B before and after modulation element showing a subpopulation B1 that is affected by the modulation. Examples of properties that may be modulated in a manner consistent with that depicted in the histograms of b include: (1) two bead populations A and B, where beads in population A have captured antigen 1 from the sample and beads in population B have not captured antigen 1. When introduced to a modulation element with antibodies that match antigen 1, only population A will be affected, leaving population B untouched. (2) a group of neutrophils with two subpopulations, one of which has high expression of a cell surface antigen. When the neutrophils pass through a modulation element with antibodies with high affinity for the cell surface antigen, two populations will be observed, with the population with high cell surface expression of the antigen shifted to the left as shown in the figure. c Traversal time through the modulation element showing four different populations. d The relationship between affinity of the particle to the bio-recognition membrane and the traversal time through the modulation element.

FIG. 6. a Parallel and serial combinations of detection/modulation elements. b Repeat use of detection elements in series. A total multiplexing number is accordingly determined by the number of modulation elements by the detection multiplexing.

FIG. 7. An exemplary preparation process for artificial beads before introduction to the system, including spherical particles having a surface molecule attached thereto, such as DNA, protein or more generally any molecule capable of being directly or indirectly connected to the surface.

FIG. 8. a Differential capture of particles for surface expression determination. b Correlation between concentration of target and number of particles captured.

FIG. 9. a Schematic illustrating the concept of stopwatching and slowing down of particles. b Histograms showing the transit times of target cells versus other cells. c Correlation between transit time and expression level of biomolecules on the surface of the particles.

FIG. 10. Schematic illustrating the process with multiple modulation elements with different receptor coatings.

### DETAILED DESCRIPTION OF THE INVENTION

In general, the terms and phrases used herein have their art-recognized meaning, which can be found by reference to standard texts, journal references and contexts known to those skilled in the art. The following definitions are provided to clarify their specific use in the context of the invention.

“Label-free” refers to the described systems and methods that provide particle property information without any need for a label. This is a particularly relevant aspect for point of care devices, including in remote locations, where use of any such label is impractical. Furthermore, added components, complexity and costs associated with reliably detecting such labels are avoided. Accordingly, label-free includes fluid samples that do not contain any optical labels such as fluorescent dyes or other tracers.

“Particle” is used broadly herein to refer to a natural, artificial, or combination of natural and artificial components of a particle complex. The particle may be characterized as generally spherical in shape, and can include a biological cell or a synthetic sphere. Typical applications of interest relate to microparticles. A microparticle refers to a particle having an average diameter that is in the micrometer scale, such as between about 1 μm and 1000 μm, or between 1 μm and 100 μm, or between 1 μm and 50 μm.

“Particle property” refers to a property of the particle that the methods and systems provided herein are characterizing. In contrast to particle parameter defined hereinbelow, this property is useful at a whole-application level. For example, the particle property in a detection assay may be the presence or absence of a type of particle, a molecule or biomarker connected to the particle or that is in the fluid sample, the robustness or detected parameter value of a particle to stimuli, including chemical, electrical or magnetic, or the concentration of a biomolecule in a fluid sample such as plasma.

“Particle parameter”, in contrast to “particle property” above, refers to a measurable and quantifiable property of a particle by a detection element and that is used to assist with the characterization of the particle property. For example, a particle size and/or type may influence an electrical parameter measured by an electrode, with each particle passing over the electrode perturbing an electric field in a confined region that is measurably detected by the electrode that is part of the detection element. Various representative examples are summarized in Table 1. This may be on a particle-by-particle basis. Particle parameter may also refer to the act of noting when the particle passes, such as by a time stamp. Such a time stamp is particularly useful for applications where a time stamp is recorded both by the up and downstream detection elements, so that an elapsed time corresponding to transit time may be calculated. In this manner, a stop-watch type of measuring is provided, with each particle transit time measured by the difference in time stamps of the first (upstream) and second (downstream) detection elements.

“Aggregate particle parameter” refers to a population of particles that has flowed past the detection element and a population-level particle parameter obtained from the plurality of particles. In this manner, the aggregate particle parameter may be considered a statistically calculated particle parameter from a plurality of individual particles and the comparison between the first and second detection elements that is a population-level determination. The methods and systems provided herein are also, however, compatible with an individual-level particle comparison, where unique individual particles are associated with the upstream and downstream detection elements. An advantage, however, of the population level comparison is that there may be a higher-throughput of particles as the comparison is instead based on population-level comparisons rather than at the individual level.

“Particle flow parameter” refers to a parameter that characterizes particle flow. Examples include transit time, velocity, particle flux, particle spacing, and various factors related thereto, including characterization of rolling velocity and adherence. Accordingly, based on any one or more of these particle and particle flow parameters, a particle property may be characterized.

“Detection element” refers to the component that is positioned fluidically adjacent to the modulation element and that detects a particle parameter as the particle flows past. Exemplary detection elements include electrodes configured to electrically detect and/or measure electrically-based particle parameters. Accordingly, the active portion of the detection element may be configured to have a fluidic portion arranged to ensure particles pass over the detection element in single file. Accordingly, the effective diameter of the fluidic portion may approach the particle diameter, such as less than two-times an average particle diameter. In this manner, single file particle flow is encouraged.

“Modulation element” refers to the component that is positioned between, in a fluidic sense, detection elements and that is capable of affecting a change in a particle, including a change in the particle itself or a flow characteristic of the particle. Accordingly, depending on the application, the modulation element can have any of a variety of configurations. With respect to detection of a molecule, polypeptide, polynucleotide, protein, or the like, including on a particle surface, the modulation element may have a functionalized surface configured to specifically interact with the to-be-detected molecule, polypeptide, polynucleotide or protein. The functionalized surface is also referred herein as a “bio-recognition membrane.” Similar to the fluid conduit portion of the detection element, the modulation element fluid conduit portion may be configured to ensure the particle has an opportunity to interact with the functionalized surface. Accordingly, at least one dimension of the fluid conduit may be configured to at least approach the size of a characteristic particle dimension, such as a channel height or diameter that is within 10×, 5× or 2× of the characteristic particle dimension. Similarly, the length of the fluid conduit portion of the modulation element may be sufficiently long so that particle settling due to gravity facilitates particle-modulation surface interaction. The fluid conduit may be, for example, circular in cross-section or have a parallel plate type geometry. For cylindrical cross-sections, an entire section of the vessel may be functionalized. For the parallel plate-type geometry, one or both of the top and bottom surfaces may be functionalized.

As used herein, “substantially single file” refers to a flow of particles such that at least 50%, at least 75%, at least 80%, at least 90%, or all the particles are individually detectable. This is a reflection that the methods and systems can tolerate some particle overlap, but it is preferred for the particle-by-particle characterization if most of the particles are in single file flow arrangement.

### Example 1: Overview of Multiplexed Label-Free Detection

The multiplexed detection of biomarkers from bodily fluids has important implications for the future of healthcare. There exists a significant paradigm shift towards emphasis on personalized and preventative medicine. For any of these concepts to become a reality, more frequent profiling of host response biomarkers is needed to: (1) understand the complex pathways leading up to disease, (2) utilize this knowledge to predict the future outcome for individual patients based on their own “biomarker fingerprint”, and (3) to use this prediction of the future to stop diseases in their tracks before they become debilitating. To achieve this, point of care devices capable of measuring many relevant biomarkers from bodily fluids are critically necessary.

The technology provided herein, for example, can facilitate point of care devices capable of profiling many different types of relevant biomarkers from blood. Most host response pathways can be monitored by tracking cell activity, cell surface proteins, plasma proteins, plasma nucleic acids, and other small molecules. The platform described herein is capable of profiling all of these entities in a single, unified platform.

Fundamentally, the technology has application for the measurement of the surface concentration of biological molecules on a spherical particle. Conceptually, there are certain similarities to systems currently offered by Luminex [1], where spherical beads are used to extract biomarkers from samples. In those systems, however, the beads are run through a flow cytometer to extract the original concentration of the target analytes. The systems and methods described herein, however, may be entirely non-optical, eliminates any need for labelling, and is much more scalable than comparable Luminex systems. These differences provide functional and fundamental advantages, including the ability to achieve a point of care device without prohibitively high costs.

Provided herein is a technology platform that can profile relevant biomarkers from whole blood by measuring the level of interaction of these particles (either cells or beads) with a modulation element functionalized with complementary antigen or antibody. To do this, two main modular elements are required: a detection element, and a modulation element.

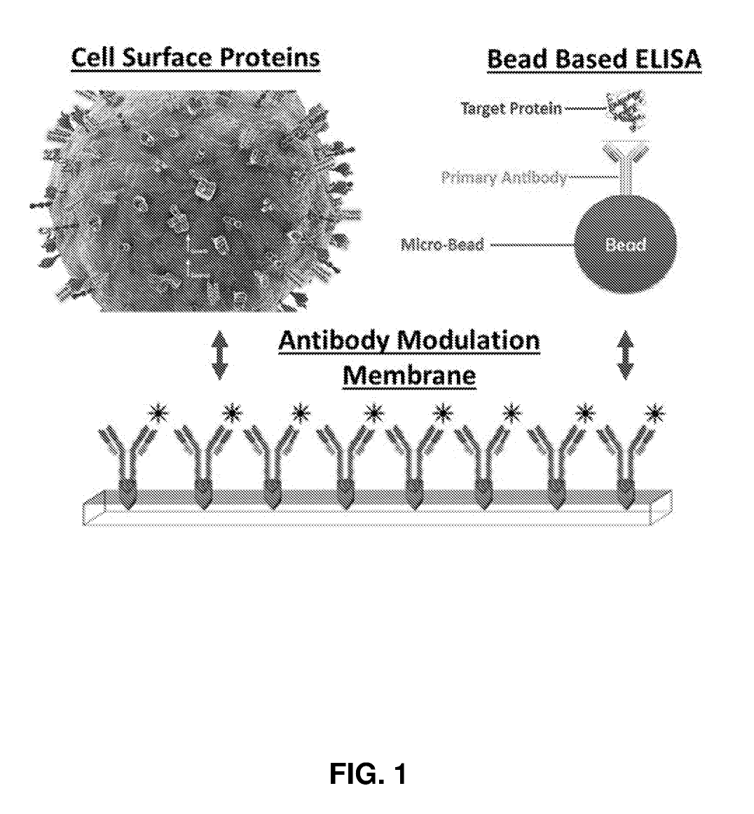

The main goal is to provide a single platform for tracking of host response pathways by detection of all relevant host response biomarkers, including cell counts, cell surface antigen expression, plasma antigens, and other plasma biomarkers such as nucleic acids or small molecules. The core elements of modulation for the technology are shown in FIG. 1. Fundamentally, a particle, which can be a cell or a microsphere, must interact with a modulation membrane which is designed with affinity to the analyte of interest. For example, leukocytes from whole blood may be the particle that express various surface proteins based on different disease states. Similarly, microspheres can be modified to a similar condition with ELISA bead kits that can anchor antibodies complementary to the antigen of interest to the surface of the bead to function as the particle. In both cases, a particle in the 5-20 μm range with varying expression levels of protein may be measured by the system.

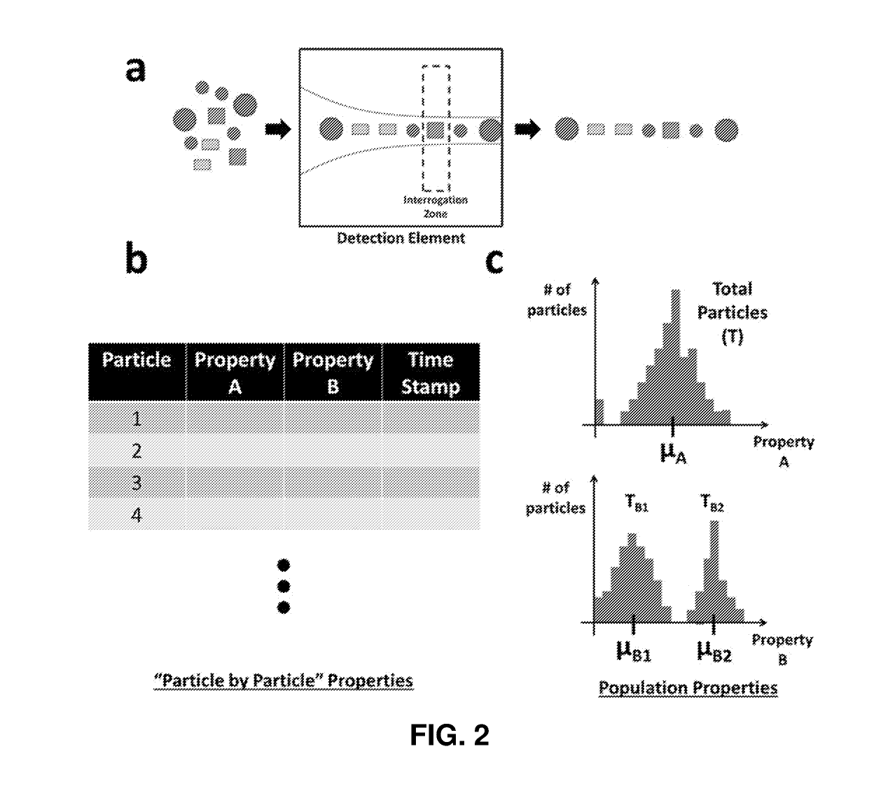

A. Detection Element: A detection element registers the presence and properties of the particles of interest on both an individual particle level and a whole population level. This is illustrated in FIG. 2. A collection of particles present in a sample is introduced into the detection element in a single file fashion. As a particle passes through an interrogation zone in the detection element, its properties and a time stamp are recorded. The recorded properties depend on the nature of the interrogation zone. For example, if an impedance-type counter, such as a coulter counter, is used for the interrogation zone, properties such as frequency dependent pulse amplitude and pulse width can be recorded. These measurements can then be used to determine intrinsic properties of the particle, such as size (impedance measurements), material properties (capacitive measurements) or dielectric/transmission properties (optical measurements). As shown in FIG. 2 panel b, these properties and a time stamp can be recorded on a particle by particle basis for all particles running through the system.

After all of the sample is run through the detection element, this data can then be summarized as population data FIG. 2 panel c). Histograms of the various measured properties can be constructed to identify total particle count, groups of particles according to separation in populations, and particle counts in these individual groups. For example, if a population of lymphocytes (7 μm-10 μm) is mixed with 15 μm beads, a size histogram of the measured population can yield a plot similar to that shown in FIG. 2 panel c (bottom), where two separate populations are clearly observed. The total count of lymphocytes plus beads, total counts of just lymphocytes, and total counts of just beads, the average response of lymphocytes, the average response or beads, and the variation in this response can all be quantified using this configuration.

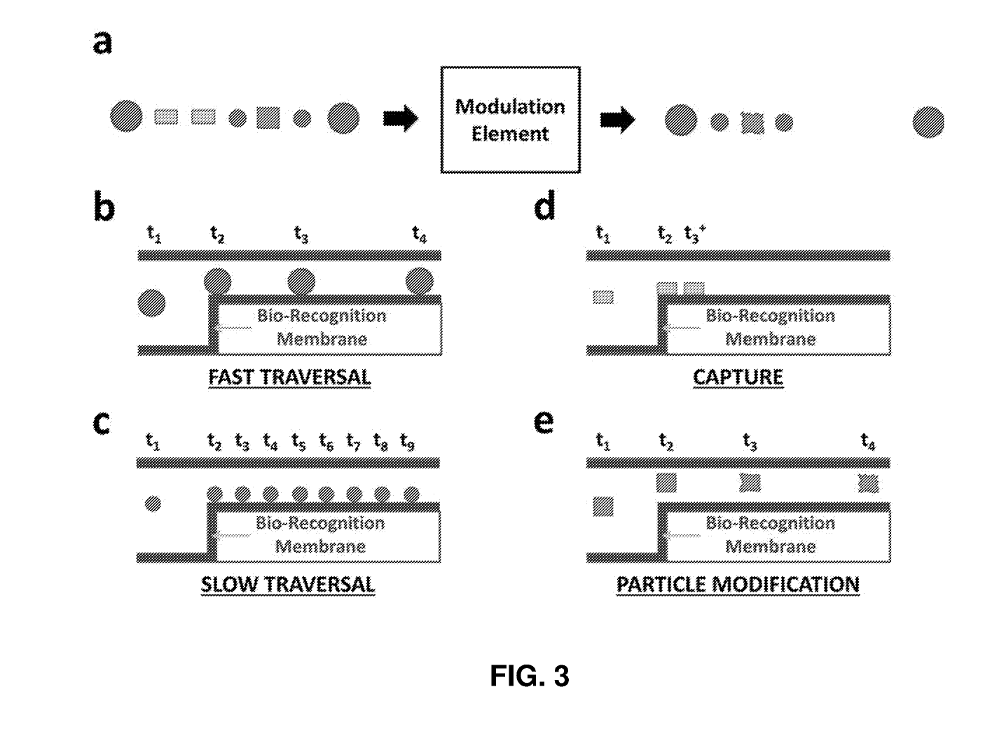

B. Modulation Element: A modulation element is provided to extract information about the molecules on the surface of the particle (FIG. 3). The modulation element comprises a normal microfluidic element that can be coated with a bio-recognition membrane that interacts with the particles as they pass through the element, in a manner equivalent to a surface coated with a target capable of binding to a counter molecule connected to the surface of the particle. Alternatively, the particle can be modified as it passes through this element with a chemical or physical process. Table 1 exemplifies a variety of modulation and detection elements for a range of applications.

As particles pass single file into the modulation element, several possible effects can occur that each provides information and characterization of a property of the particle. The particle could pass more or less unaffected through the modulation element if the biomarkers on the surface of the particle have very little affinity to the bio-recognition membrane in the modulation element (FIG. 3 panel b). This indicates an absence of a molecule capable of specific interaction with the counter-target on the membrane. The particle could be slowed as it rolls on the surface of the bio-recognition membrane if the surface biomolecules have high affinity to the membrane (FIG. 3 panel c). In this case, the passage time through the modulation element is increased as the on/off binding events slow the particle compared to an equivalent particle that does not interact with the membrane targets. The particle could also be completely captured and arrested with respect to fluid flow by the bio-recognition membrane (FIG. 3 panel d). In this case, the particle will not exit the modulation element. Finally, the particle can be chemically or physically modified in the modulation element (FIG. 3 panel e), with various examples provided in Table 1.

Information about the particles before entering the modulation element and information about the particles after the modulation element can be compared to extract out the relevant properties about the particle. One example is the presence/absence and/or surface concentration of the biomolecules of interest on the particle.

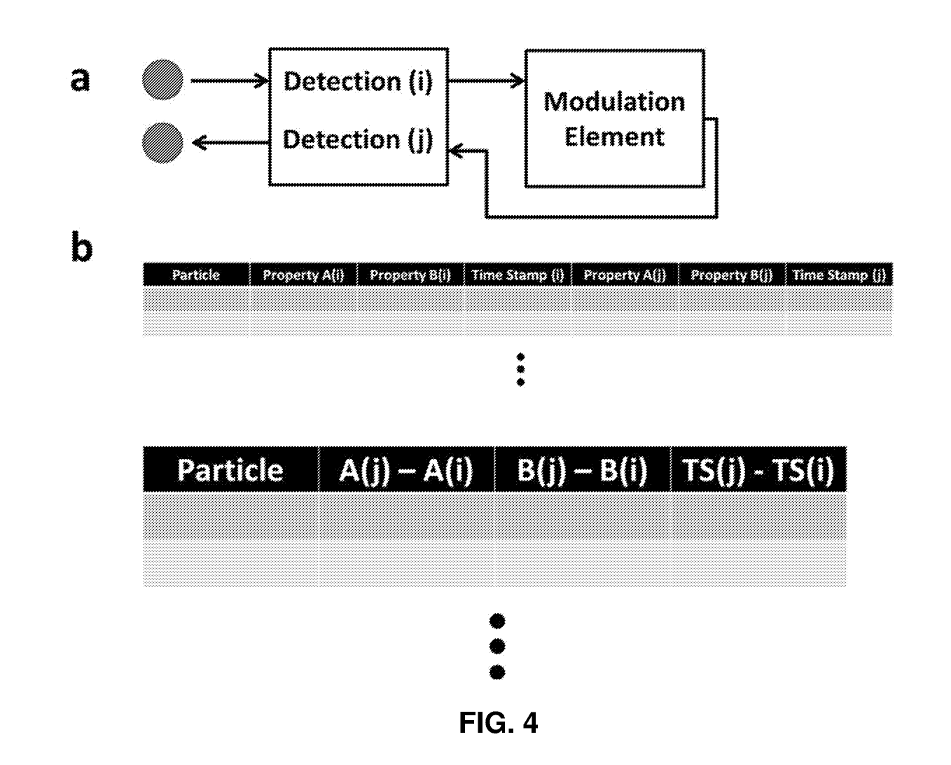

C. Combined Elements: Detection elements and modulation elements can be combined to extract the desired particle properties. One relatively simple embodiment is illustrated in FIG. 4. Here, particles pass through a detection element for measurement, as illustrated by Detection(i) for the detection before introduction to the modulation element. As described previously, properties for these particles and a time stamp is recorded on an individual basis (Property A(i), Property B(i), Time Stamp (i)). The particles are then passed through the modulation element, immediately followed by passage through a detection element for Detection(j), including a detection element that is the same detection element for the detection prior to introduction to the detection element. Appropriate dilution of the particles is maintained so that two particles are unlikely (e.g., less than 50% likelihood) or never in the detection element simultaneously. This can be mathematically determined based on the detection element area, flow rate and particle concentration, so that the particle flux to the detection element ensures that not more than one particle is over the detection element at any given time. In other words, the particle concentration is not more than one particle per detection element interrogation area or corresponding fluid volume. In an exemplary embodiment, a detection element interrogation area corresponds to a typical cross-sectional area of an aperture, also referred to as a Coulter aperture, and may be about 225-10,000 μm², with a corresponding volume of about 3.4-10,000 pL. The second set of detection data is then recorded, also on an individual level (Property A(j), Property B(j), Time Stamp (j)). As desired, feedback controls may be employed so that as the detection elements detect a particle flux that is too high or too low, fluidic controllers may be engaged to ensure a desired or optimum particle flux is maintained. The fluidic controllers may be a combination of pumps and valves upstream of the system, where one fluid stream without particles mixes with another fluid stream that contains particles, thereby controlling particle flux introduced to the upstream detection element. Accordingly, any of the methods provided herein may further comprise selecting an optimal particle flux density, continuously determining particle flux density in the first and/or second detection element, and adjusting particle flux density in the conduit by controlling fluid mixing upstream of the first detection element.

The recorded properties can then be compared on a particle-by-particle basis (FIG. 4 panel b, bottom table). The difference in the measured properties due the modulation element can yield information about the particle's interaction with the modulation element. For example, longer passage times (TS(j)-TS(i)) indicate higher affinity of the particle's surface molecules to the bio-recognition membrane. Particles may also be missing in the second detection, indicating that the particle is captured in the modulation element.

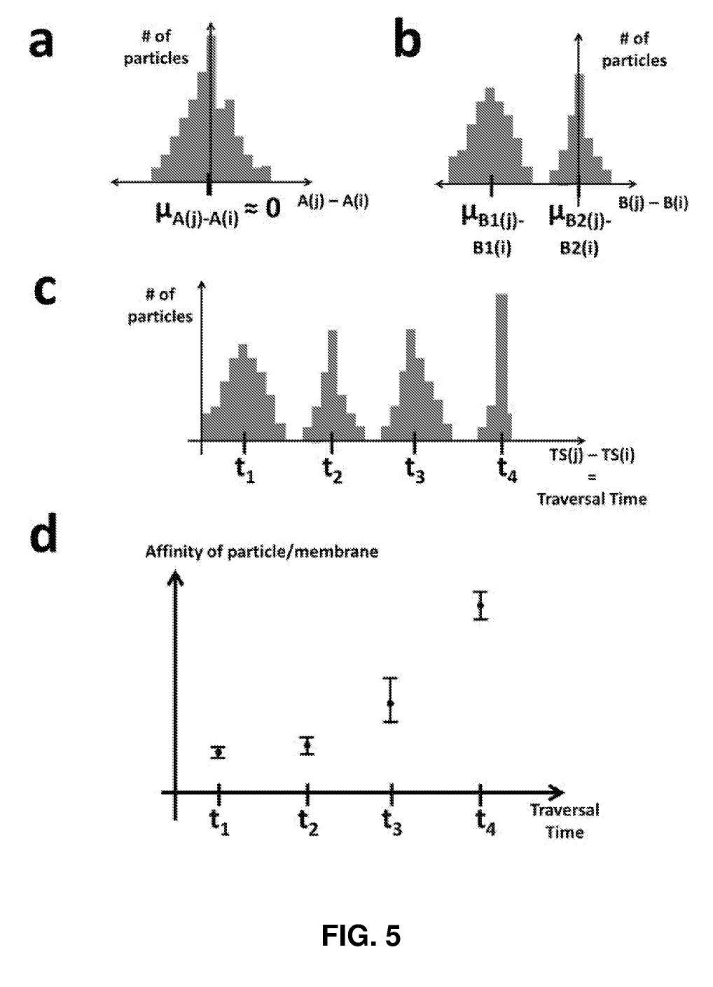

These differences in recorded properties due to the modulation element can also be plotted on a population level. An exemplified difference in property A (A(j)-A(i)) is shown in FIG. 5 panel a. Here, the modulation element has little effect on the particle population (the mean of the difference is close to 0). In FIG. 5 panel b, an example of a shift in part of the population due to the modulation element is illustrated. In this case the B2 population is unaffected by the modulation element, but the B1 population is clearly affected by the modulation. Similar histograms can also be plotted for the residence time (TS(j)-TS(i)) in the modulation element or for the difference in subpopulation or total counts of the particles before and after modulation. If several population of particles (each with different affinities to the bio-recognition membrane) exist in the sample, a histogram such as that shown in FIG. 5 panel c could result. Here, four separate populations with distinct residence times are shown. With appropriate calibration experiments, this data can be utilized to back out the affinity of the particle to the bio-recognition membrane (FIG. 5 panel d) and thus the concentration of the biological molecules on the particles of interest.

D. Scalability of the Approach: The approach described above is scalable and can also be multiplexed. The platform is capable of the detection of proteins, DNA, and cells—including all from the same device and even the same assay. For example, the use of ELISA beads for the detection of plasma biomarkers allows the use of a single platform for all different types of analytes. In all cases, the system can measure the relevant biomarkers by extracting the surface expression of biomolecules on the surface of a particle, including a spherical particle such as cells and/or beads.

One of the key advantages of the approach is the scalability to provide multiplexing of biomarkers capability when compared to optical techniques. Optical techniques require a different color fluorophore for each new target of interest so that each target can be optically distinguished. This typically requires an additional excitation laser for fluorophores that have different excitation wavelengths, as well as additional emission filters for appropriately detecting emitted light at an appropriate wavelength, thereby significantly increasing complexity for each added multiplexed target. Each additional increase in complexity in technology enormously decreases the feasibility of a point of care device.

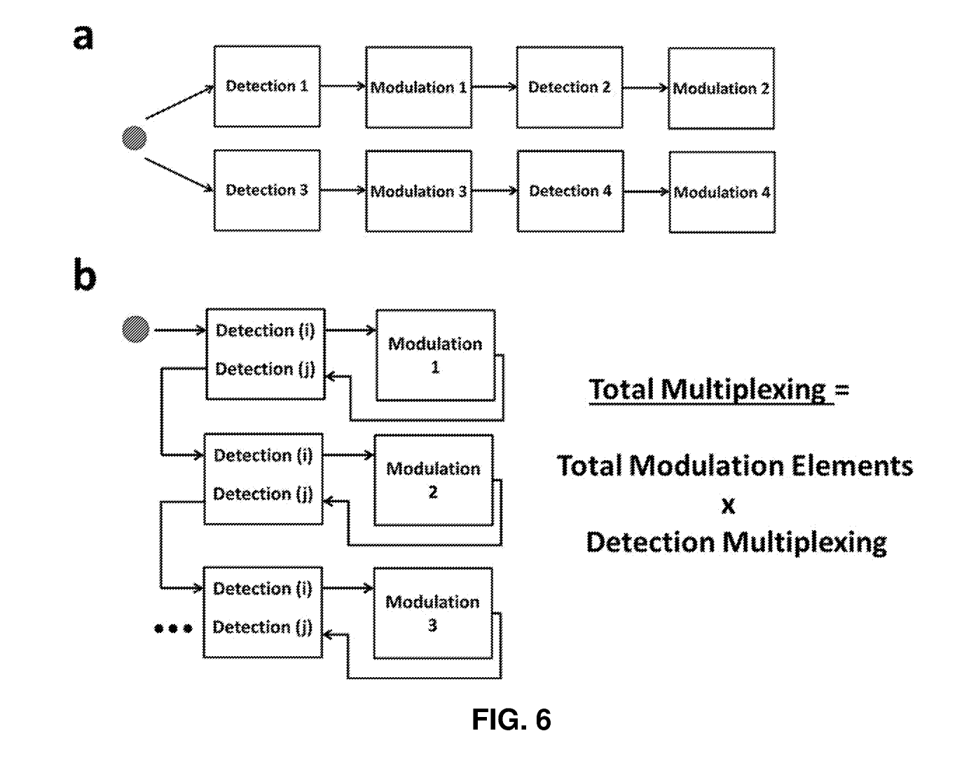

With this platform, multiplexing of targets is achieved through the spatial use of different modulation elements in a linear or parallel fashion (FIG. 6); each element functionalized with a different bio-recognition membrane for different multiplexed targets. In this fashion, instead of modifying each particle differently for different analytes, the same particle is interrogated for multiple analytes by use of distinct modulation elements tailored to different analytes. This is a fundamental difference between this platform and any optical platform that purports to provide a feasible point of care device for tracking of many biomarkers from blood.

As shown in FIG. 6, detection and modulation elements can be combined in parallel and in series in many configurations. Detection elements can be re-used (FIG. 6, panel b) to reduce complexity of the system. The total multiplexing capability can be calculated by the total number of modulation elements multiplied by the inherent capability for detection elements to multiplex. For example, if the detection element is inherently capable of identifying 3 separate populations (based on properties of the particles in the interrogation zone of the detection element such as different particle sizes providing a different electrical measure) and 5 modulation elements are used (each with a different target analyte in mind), a total of 15 multiplexed target entities can be queried.

E. Exemplary Applications of the Platform:

1. Differential Counting of Capture: One embodiment of the platform is the use of a detection module, followed by a modulation element, followed by another detection module. A version of this, where leukocytes are counted using an impedance counter, followed by specific capture in a capture chamber functionalized with CD4 or CD8 antibodies, followed by a second count using another impedance counter, is described [2-5]. Accordingly, any of the systems and methods specifically exclude the capture and counting embodiments described or suggested in any one or all of publications [2-5], and each of [2-5] are specifically incorporated by reference herein for the capture and counting embodiments described therein.

Examples of methods not previously described include the use of differential counting before and after a capture chamber for: (i) Quantification of surface expression of antigens on the surfaces of cells by counting the number of cells captured in a capture chamber. This can include multiple capture steps with varying conditions to modulate the number of captured cells (such as antibody density in the chamber, shear stress used, incubation time, etc.); and (ii) Quantification of surface expression of biomolecules on the surfaces of beads by counting the number of beads captured in a capture chamber with the relevant coating of complementary molecules. Again, this can include multiple capture steps with varying conditions as mentioned in (i).

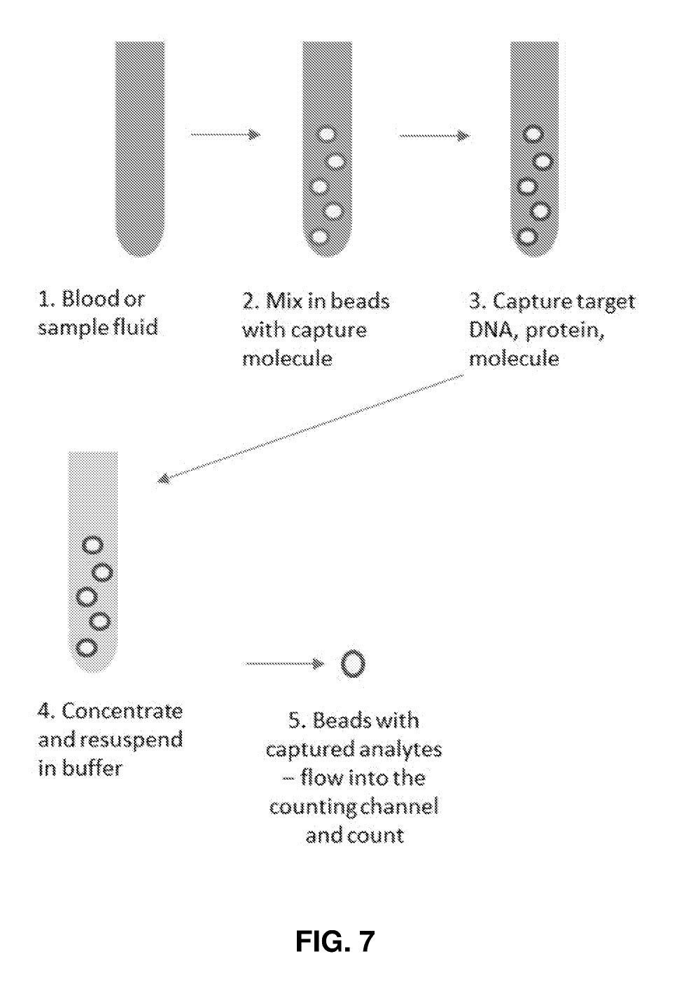

These two methods are similar, except for pre-processing steps in the case of beads. FIG. 7 illustrates a possible steps for the pre-processing of beads to form a particle with a concentration of biomolecules on its surface ready for introduction to any of the systems described herein. Beads pre-coated with primary antibodies from an ELISA kit are incubated in the sample to capture the target biomarkers (DNA, proteins, or small molecules). The beads are then recovered and re-suspended in a buffer prior to introduction to the modulation elements.

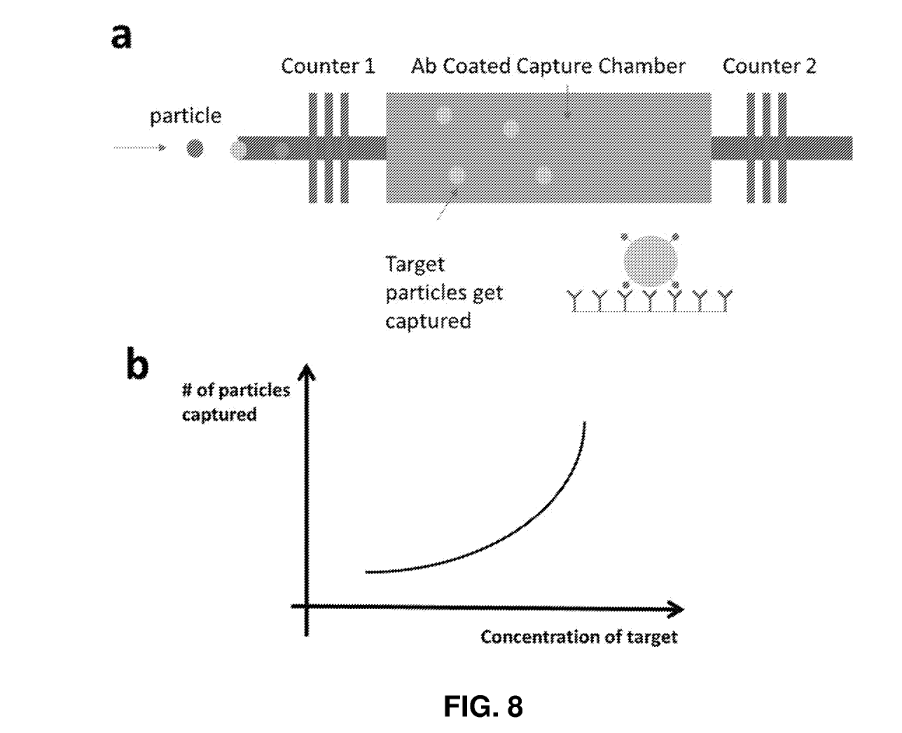

At this stage, the system is the same whether the particle is a non-naturally occurring bead or a biological cell. FIG. 8 illustrates capture of the particles to calculate the surface concentration of biomolecules. The particles flow past the first detection element, are counted, and then flow through a modulation element, such as a capture chamber. Depending on the surface concentration of the target analytes, different numbers of particles are captured in the capture chamber. The number of captured beads is correlated to the surface expression of proteins on the beads [6]. In this system, this number is measured by subtracting the difference between the exit and entrance counts. With appropriate calibration curves, the concentration of the target analyte on the surface of the particle can thus be determined.

2. Biomolecular Concentration Level Determination Via Transit Time Through a Functionalized Channel: In this example, particles (beads or cells) can traverse through a modulation element, such as a functionalized channel, at different speeds depending on the characteristics of the target analyte on the particle and surface that comprises a bio-recognition element of the modulation element. The transit time for the particles is then proportional to the affinity of the particle to the bio-recognition membrane, and thus also to the concentration of the target analytes coating the particles.

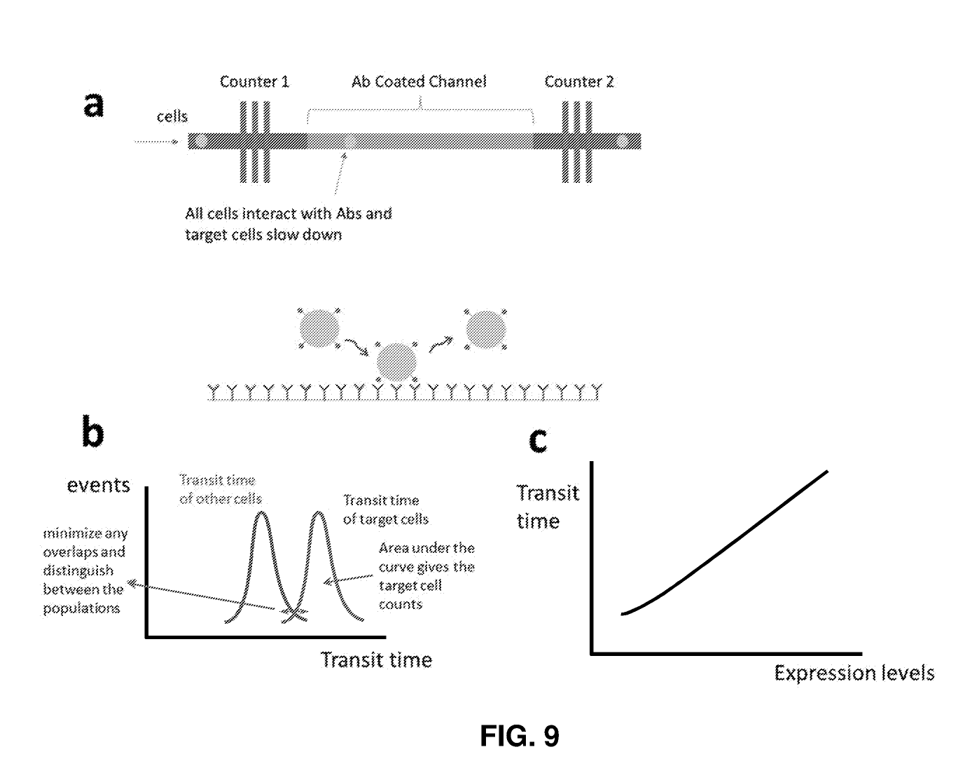

The difference in velocity between a free-flowing particle and a flowing particle that interacts with the vessel wall, such as mediated by receptor-ligand interactions, is characterized as a rolling velocity. Rolling of biological cells on surfaces have been demonstrated—the speed of which is proportional to the surface expression of protein on the outside of the particle [7-9]. This platform can utilize this concept with two detection elements or “modules” to track the particles on an individual basis using a “stopwatching algorithm” (FIG. 9). As the beads or cells pass the first detection module, a time stamp is recorded for each particle. The particle then interacts with sidewalls and obstacles of the microfluidic channel that are coated with complementary antibodies or nucleic acids to the molecules on the surface of the particle. Due to this interaction, the speed of the particle will be modulated. As the particle exits the channel and passes past the second detection module, another time stamp is recorded. From these two stamps, a transit time for each particle can be recorded. The methods and systems are compatible with a range of transit times (t), lateral flow dimensions (L), flow rates (Q), and particle flux density (F; number of particles per second), such as t between 0.1 and 10 (seconds), L between 0.1 and 50 (mm), Q between 0.01 and 1 (μL/s) and F between 0 and 100 particles per second From the transit time, the affinity of the particle to the antibodies in the channel is determined and, thus, the concentration of biomolecules on the surface of the particle determined.

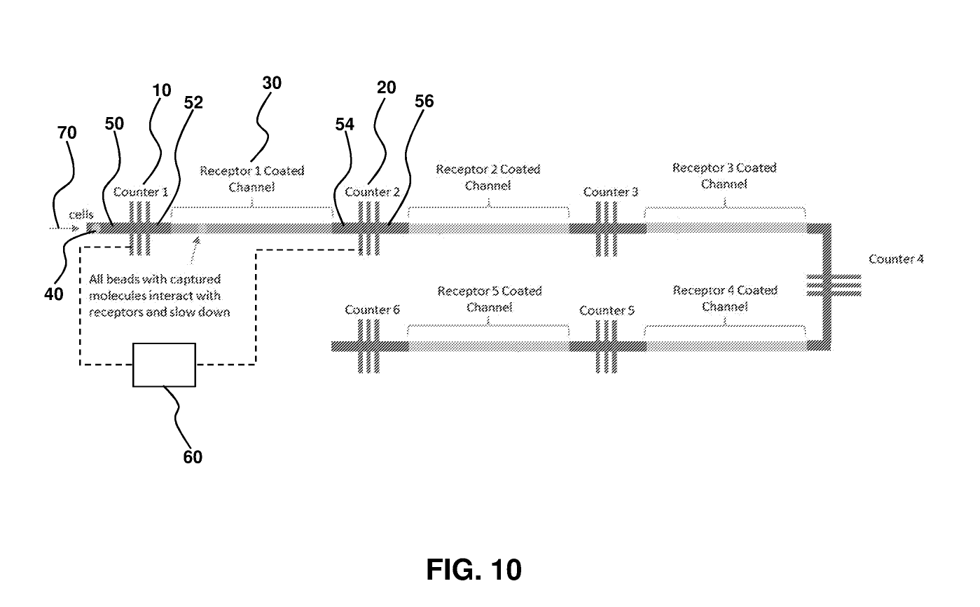

3. Multiplexing with Multiple Modulation Elements: FIG. 10 illustrates an approach for multiplexing of detection elements. This involves many detection and modulation elements implemented in series. In the example illustrated in FIG. 10, there are five different modulation zones, each with a different receptor for targeting a different biomolecule on the surface of the particles. This approach allows for the following: (i) Multiplexing for 5P total biomolecular targets, where P is the number of targets that can be differentiated based on size or capacitive properties using a single counter; (ii) Full co-expression for all combinations of the 5P targets; (iii) Capability to run beads (plasma biomarkers), cell counts, and cell surface proteins all from the same platform.

Relevant components of the system include detection elements 10 and 20 that are arranged upstream and downstream, respectively, of modulation element 30. Adjacent detector elements are separated by a modulation element. In this example, the modulation element has a functionalized surface corresponding to a receptor against a target on the particle 40. The particle 40 may be a cell. Fluidic conduits 50 52 54 56 may be selected to have a dimension corresponding to the size of the cells. This helps facilitate and ensure single-file flow. An electronic system 60 is electrically connected to the detection elements 10 and 20, as indicated by the dashed lines, so as to provide recording and comparison ability with respect to parameters detected by the detection elements. For simplicity, the electrical connections with respect to the other detection elements are not illustrated. Pump 70, such as a microfluidic pump, indicated by the arrow into the fluid conduit 50 provides the ability to selectively control flow rate and particle flux through the system. As desired, additional fluidic components are incorporated into the system, including in and around a region of the pump 70. For example, multiple separate flow conduits may connect to provide a desired flux of particles 40 to ensure at any one time, a single particle is provided to detection element 10, and other downstream detection elements, labeled as Counter 2-5. The separate flow conduits may correspond to a first conduit containing particles and a second conduit containing suspension media, wherein the relative flow rates in the conduits are controlled to achieve a particle flux introduced to detection element 10 that is between a user-selected particle flux range. The user-selected particle flux range, for example, is selected to ensure only one particle is detected by detection element 10 at any given time. Depending on the type of physical parameter being measured, the detection element may be an electrode or a plurality of electrodes. As desired, the modulation elements, indicated as coated channels having different receptors, may be replaceable, such as by positioning the modulation elements in a removable cartridge.

To summarize, the described platform has the following fundamental advantages over other technologies currently being developed for similar applications: (i) A single, unified platform for all relevant host biomarkers, including cell counts, expression of cellular surface proteins, plasma proteins, nucleic acids, and small molecules; (ii) A much more scalable approach for multiplexing of many biomarkers from the same device when compared to optical techniques by the use of modulation zones for spatial multiplexing; (iii) Elimination of the need for all optical components and labelling process, which significantly increases the feasibility of cost efficient point of care devices.

### REFERENCES FROM THE EXAMPLE

1. M. F. Elshal and J. P. McCoy, Jr., “Multiplex Bead Array Assays: Performance Evaluation and Comparison of Sensitivity to ELISA,” Methods 38 (4), 2006.
2. N. N. Watkins, B. M. Venkatesan, M. Toner, W. Rodriguez, and R. Bashir, “A Robust Electrical MicroCytometer with 3-Dimensional Hydrofocusing or Portable Blood Analysis,” Lab on A Chip 9 (3177), 2009.
3. N. N. Watkins, S. Sridhar, X. Cheng, G. D. Chen, M. Toner, W. Rodriguez, and R. Bashir, “A microfabricated electrical differential counter for the selective enumeration of CD4+T lymphocytes,” Lab On A Chip 11 (1437), 2011.
4. N. N. Watkins, U. Hassan, G. Damhorst, H. Ni, A. Vaid, W. Rodriguez, and R. Bashir, “Microfluidic CD4+ and CD8+T lymphocyte counters for point-of-care HIV diagnostics using whole blood,” Science Translational Medicine 5 (214), 2013.
5. PCT Application No. PCT/US2011/060041, “Counting particles using an electrical differential counter”. Xuanhong Cheng, Rashid Bashir, Mehmet Toner, Aaron Oppenheimer, William Rodriguez, Nicholas Watkins and Grace Chen. Priority date: Nov. 9, 2010. Publication date: May 18, 2012. Filed in: United States, Europe, China, and South Africa.
6. J. Mok, M. N. Mindrinos, R. W. Davis, and M. Javanmard, “Digital microfluidic assay for protein detection,” Proceedings of National Academy of Sciences 111 (6), 2013.
7. S. Choi, J. M. Karp, and R. Karnik, “Cell Sorting by deterministic cell rolling,” Lab on a Chip 12 (1427), 2012.
8. D. J. Sherman, V. E. Kenanova, E. J. Lepin, K. E. McCable, K. Kamei, M. Ohashi, S. Wang, H. Tseng, A. M. Wu, C. P. Behrenbruch, “A differential cell capture assay for evaluating antibody interactions with cell surface targets,” Analytical Biochemistry 401, 2010.
9. A. W. Greenberg and D. A. Hammer, “Cell Separation Mediated by Differential Rolling Adhesion,” Biotechnology and Bioengineering 73 (2), 2001.

### Example 2: Drug Characterization and Efficacy Evaluation

The methods and systems have a number of practical applications, including drug screening applications to evaluate effectiveness of therapeutic candidates. One application of such a screen is for cancer applications. In particular, the systems provided herein can assess mediator secretion response and surface protein expression response. The basic methodology is the biological cell/sample is passed through an initial modulation element which presents an antigen or biochemical modulator to the cell/sample. As desired, an incubation period may be included to ensure sufficient time for a desired cascade in the cell or other biological material. The response of a cell to the modulation element is, depending on the resultant cascade events, one or more of stimulation or inhibition, mediator release, and/or surface protein expression. This list is representative, as other morphological changes are compatible with the instant processes and devices. Any induced change may then be measured by a second element, which is a sensor-modulator-sensor element described herein (see, e.g., Table 1).

An exemplary flow-chart summary for an application may include:
Step 1. A cell is passed through a detection zone 1
Step 2. The cell passes through modulation zone 1, which slows down the cell, dependent on a surface property of the cell.
Step 3. The cell passes through detection zone 2, using zone 2—zone 1 difference, a property of the cell is measured.
Step 4. The cell passes through modulation zone 2, where a chemical stimulus is applied (e.g., a drug that is being screened for an efficacy or desired cell response)
Step 5. The cell passes through a detection zone 3 for first measurement
Step 6. The cell passes through modulation zone 3, which slows down the cell based on the same surface property of the cell as modulation zone 1
Step 7. The cell passes through detection zone 4 for final measurement

In the above-referenced application, detection zones 2 and 1 provide a measure of the initial surface property, whereas detection zones 4 and 3 provide a measure of the final changed surface property, arising from the chemical stimulus. Accordingly, a comparison of the detection from zones 2 and 1 to the detection from zones 4 and 3 provides useful characterization of the chemical stimulus, particularly chemical efficacy.

TABLE 1
Exemplified systems summary:

| | | | | Detector |
|---|---|---|---|---|
| | | Modulation | Modulated | Element(s)/particle |
| Application | Particle Type | Element | Property | parameter |
| Detection | Microparticle | Bio-recognition | particle flow | electrical detector |
| and/or | having cell surface | membrane | velocity - including | that records time for |
| quantification of | molecules, | | relative to bulk fluid | particle to transit |
| surface | including biological | | flow rate | modulation element |
| molecules | cells and/or | | | |
| | synthetic | | | |
| | microspheres | | | |
| Detection of | Microparticle that | Bio-recognition | particle flow | electrical detector |
| molecules, | is surface | membrane | velocity - including | that records time for |
| including | functionalized to | | relative to bulk fluid | particle to transit |
| plasma | bind to the plasma | | flow rate | modulation element |
| analytes, | molecule | | | |
| exogenous | | | | |
| analytes | | | | |
| (pathogens, | | | | |
| antigens, toxins, | | | | |
| drugs), fluid, | | | | |
| such as is urine, | | | | |
| CSF, saliva | | | | |
| Detection of | Biological cell | Conduit geometry, | physical | electrical detector |
| particle stiffness | | such as size | deformation of | that records time for |
| | | through which | particle to transit | particle to transit |
| | | particle flows | the modulation | modulation element |
| | | | element, with stiffer | |
| | | | particles taking | |
| | | | more time to transit | |
| Multiplexing | Multiple | Plurality of | particle flow | Multiple detector |
| | populations and/or | modulation | velocity, with each | pairs to record |
| | sub-populations | elements, each | modulation element | particle transit time |
| | | modulation element | affecting velocity | across individual |
| | | having a different | based on a | modulation |
| | | bio-recognition | different surface | elements |
| | | membrane | molecule | |
| Detection of | Biological cell (e.g. | Membrane with | Aggregation of | Size and/or quantity |
| endogenous | platelet) | biological stimulator | particle | of particle |
| molecule | | | | aggregates |
| activity | | | | |
| Electrolyte | Microparticle | Selective ion- | Bulk fluid | Electrical detector |
| concentration | | binding element | conductivity or | |
| | | | microparticle | |
| | | | electrical signature | |
| Mediator | Biological | Biochemical | Mediator release | electrical detector |
| secretion | cell/microparticle | stimulator or | | that records time for |
| response | | antigen | | particle to transit |
| | | | | secondary |
| | | | | modulation element |
| Surface protein | Biological | Biochemical | Cell surface | electrical detector |
| expression | cell/microparticle | stimulator or | expression | that records time for |
| response | | antigen | | particle to transit |
| | | | | secondary |
| | | | | modulation element |
| Characterization | Biological cells | Conduit geometry, | Chemical | Electrochemical or |
| of chemically | treated with certain | such as size | modification | electrical detection |
| modulated | chemical reagents | through which | | |
| particles | | particle flows | | |
| Detection of | Modulated Cells | Conduit geometry, | Changes in the | Magnetic e.g. GMR |
| magnetic | with magnetic | such as size | resistance | Sensing |
| particles | particles or | through which | proportional to the | |
| | individual magnetic | particle flows | particles | |
| | particles | | | |
| Characterization | Biological cell | Conduit geometry, | Refractive index | Optical microscopy |
| of Intracellular | | such as size | | |
| processes | | through which | | |
| | | particle flows | | |
| Characterization | Biological cell | Conduit geometry, | Changes in the | Electrical detection |
| of cell's | | such as size | impedance signal | based on probing |
| components | | through which | because of intrinsic | cells at multiple |
| (nucleus, | | particle flows | dielectric properties | frequencies |
| plasma/nucleus | | | of the un- | |
| membranes) | | | modulated or | |
| etc. | | | modulated cells | |
| Detection of | Biological cell or | High speed camera | Particle size, | Optical, based on |
| particles | Microparticle | | velocity, and | Image analysis |
| | | | physical | |
| | | | deformation | |
| Detection of | Microparticle | Mass sensor - A | Particle mass - | Resonance based |
| individual or | having cell surface | pedestal geometry | including absolute | mass sensor |
| modulated | molecules, | | OR increased | |
| particle | including biological | | modulated particle | |
| | cells and/or | | mass | |
| | synthetic | | | |
| | microspheres | | | |

### STATEMENTS REGARDING INCORPORATION BY REFERENCE AND VARIATIONS

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

When a group of substituents is disclosed herein, it is understood that all individual members of that group and all subgroups, are disclosed separately. When a Markush group or other grouping is used herein, all individual members of the group and all combinations and subcombinations possible of the group are intended to be individually included in the disclosure.

Every formulation or combination of components described or exemplified herein can be used to practice the invention, unless otherwise stated.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

We claim:

1. A label-free method for characterizing a property of a particle suspended in a fluid sample, the method comprising the steps of:
flowing a fluid sample containing particles across a first detection element, wherein the particles flow in substantially single file across the first detection element;
detecting with the first detection element a particle parameter for at least a portion of the particles that pass the first detection element;
flowing the particles from the first detection element to a first modulation element, wherein the first modulation element effects a change in the particle parameter of the particles flowing past the first modulation element;
flowing the particles from the first modulation element across a second detection element, wherein the particles flow in substantially single file across the second detection element;
detecting with the second detection element the particle parameter for the at least a portion of the particles that pass the second detection element, wherein the particle parameter detected by the second detection element has a value that is different than a value of the particle parameter detected by the first detection element;
comparing the particle parameter detected by the first detector with the particle parameter detected by the second detector; thereby characterizing the particle property;
wherein the particle property is selected from the group consisting of:
biomolecule presence on a surface of the particle;
biomolecule surface concentration on a surface of the particle;
biomolecule presence in the fluid sample; and
biomolecule concentration in the fluid sample.

2. A label-free method for characterizing a property of a particle suspended in a fluid sample, the method comprising the steps of:
flowing a fluid sample containing particles across a first detection element, wherein the particles flow in substantially single file across the first detection element;
detecting a first particle parameter for each particle that passes the first detection element to obtain a first aggregate particle parameter for a plurality of particles that pass the first detection element;
flowing the particles from the first detection element to a first modulation element, wherein the first modulation element effects a change in a property of the particles of the particles flowing past the first modulation element;
flowing the particles from the first modulation element across a second detection element, wherein the particles flow in substantially single file across the second detection element;
detecting a second aggregate particle parameter for each particle that passes the second detection element to obtain a second aggregate particle parameter for a plurality of particles that pass the second detection element;
comparing the first aggregate particle parameter with the second aggregate particle parameter; thereby characterizing the particle property;
wherein the particle property is selected from the group consisting of:
biomolecule presence on a surface of the particle;
biomolecule surface concentration on a surface of the particle;
biomolecule presence in the fluid sample; and
biomolecule concentration in the fluid sample.

3. The method of claim 1 or 2, further comprising the steps of: repeating the flowing steps for one or more additional detection elements and one or more modulation elements to obtain one or more additional particle parameters or aggregate particle parameters and particle properties, thereby providing a multiplex characterization for a plurality of particle properties.

4. The method of claim 3, wherein the additional detection and modulation elements are provided in a parallel configuration, a series configuration, or a combination of parallel and series configuration.

5. The method of claim 1 or 2, wherein at least one particle property provides information about a biomarker that is a receptor on a surface of the particle.

6. The method of claim 1 or 2, wherein the comparing step comprises determining:
a time elapsed between the particles that pass the first detection element and the particles that pass the second detection element; or
particle flux or spacing;
thereby obtaining a measure of a particle transit time through the modulation element and non-optically characterizing the particle property.

7. The method of claim 1, wherein the detection element detects a physical property of the particle selected from the group consisting of: an electrical property, a magnetic property, and a mechanical property; wherein a change in the detected physical property between the first and second detection element provides the particle property characterization.

8. The method of claim 2, wherein the detection element detects a physical property of the particle selected from the group consisting of: a mechanical property; and a magnetic property; wherein a change in the detected physical property between the first and second detection element provides the particle property characterization.

9. The method of claim 1, wherein the detection element comprises an electrode to detect a change in an electrical property when a particle passes the detection element.

10. The method of claim 1 or 2, wherein the first and second detection elements are a common detection element.

11. The method of claim 1 or 2, wherein the first and second detection elements are different detection elements.

12. The method of claim 1 or 2, wherein at least one detection element is configured to distinguish a plurality of particle populations.

13. The method of claim 12, wherein the plurality of particle populations are distinguished based on an electrical property, including a change in impedance as a particle passes the detector, with a first population of particles associated with a first average impedance value and a second population of particles associated with a second average impedance value.

14. The method of claim 2, wherein the first or second aggregate particle parameter is selected from the group consisting of: impedance, resistance, current, transit time, velocity, refractive index, viscosity, a magnetic parameter, a mechanical parameter such as stiffness, and a property of a constituent of the particles including a nucleus of a biological cell.

15. The method of claim 1 or 2, wherein the detection element has an interrogation zone in which the particle parameter or the first or second aggregate particle parameter is measured.

16. The method of claim 1 or 2, wherein the modulation element comprises:
a plurality of modulation element surface-bound targets that specifically bind to a counter-analyte on a surface of the particle, wherein the binding results in particle adherence to a surface of the modulation element or particle rolling over the surface of the modulation element;
a geometry configured to assess a particle physical parameter, such as stiffness, viscosity, density, size, refractive index, charge; and/or
a chemical agent to modify a particle characteristic.

17. The method of claim 1 or 2, wherein the modulation element comprises a plurality of surface-bound targets selected from the group consisting of:
a polypeptide sequence;
a polynucleotide sequence;
a protein;
an antibody;
an antigen; and
a chemical substance having activity for a biomolecule of interest.

18. The method of claim 1 or 2, wherein the modulation element generates a modulation force on the particle, the modulation force selected from the group consisting of: an antibody affinity; an optical force; a dielectrophoretic force; a lateral flow force; a microfluidic force generated by a fluidic geometry of the modulation element; and a chemically-generated force.

19. The method of claim 1, wherein the modulation element provides one or more of:
decrease in a velocity of the particle;
adherence of the particle to a surface of the modulation element; or
a modification of the particle.

20. The method of claim 1 or 2, wherein the particle is selected from the group consisting of one or more of a biological cell; a microsphere; a charged species; a protein; a polypeptide, DNA, RNA, a polynucleotide; an antibody; and an antigen.

21. The method of claim 20, wherein the particle is a biological cell from a blood sample.

22. The method of claim 21, wherein the particle is a leukocyte.

23. The method of claim 1 or 2, wherein the particle has an average diameter of between 5 μm and 25 μm.

24. The method of claim 1 or 2, further comprising diluting the fluid sample to avoid simultaneous particle detection by the first detection element or the second detection element.

25. The method of claim 1 or 2, wherein the particle comprises: a biomaterial isolated from a biological sample; or a material that specifically captures a biomaterial from a biological sample.

26. The method claim 1 or 2, wherein there is a plurality of distinct particle populations, and the method characterizes a particle parameter for each of the distinct populations.

27. The method of claim 1 or 2, wherein the particle property biomolecule is selected from the group consisting of: a cell surface receptor; plasma proteins, plasma nucleic acids, small molecules, a biomaterial released from a lysed cell; a bacteria, a virus; mRNA, and DNA.

28. The method of claim 1 or 2 used in an application selected from the group consisting of one or more of: particle counting; particle sorting; surface protein expression; plasma protein level measurement; nucleic acid detection; small molecule detection; particle motility; co-expression detection of multiple biomolecules; expression of plasma proteins or nucleic acid within a biological cell; electrolyte characterization;
and quality control.

29. The method of claim 1 or 2, wherein the modulation element is selected to provide an assessment of: cell activity; cell surface protein; plasma proteins; and/or plasma nucleic acids.

30. The method of claim 1 or 2 used in a point of care device.

31. The method of claim 1 or 2, used to measure cell surface antigen expression.

32. The method of claim 3, to measure co-expression of a plurality of cell surface markers.

33. The method of claim 1 or 2, further comprising the step of generating histograms of detected particles as a function of elapsed time between detection of the particle parameter with the first and second detection elements or the first and second aggregate particle parameters.

34. The method of claim 33, wherein the comparing step comprises determining a difference between the particle parameters detected by the first and second detection elements or the first aggregate particle parameter and the second aggregate particle parameter, and plotting a histogram of the difference for the particles in the fluid sample.

35. The method of claim 1 or 2 that provides a total multiplexing number that is the product of the total number of modulation elements and the total number of populations distinguished by the detection elements, wherein the total multiplexing number is greater than or equal to 6.

36. The method of claim 1 or 2, further comprising the step of optimizing the modulation element to control a number of captured particles by the modulation element.

37. The method of claim 36, wherein the optimizing comprises one or more of: selecting a shear force at the modulation element wall; incubating particles in the modulation element for an incubation time; or selecting a target element density on the modulation element wall.

38. The method of claim 1 or 2, for quantifying surface expression of biomolecules on a particle surface.

39. The method of claim 38, wherein the quantifying is by counting a number of particles captured by the modulation element having a surface coating of target molecules specific for the biomolecules on the particle surface.

40. The method of claim 38, wherein the particle is a bead and the biomolecules on the bead surface correspond to a biomaterial isolated from a biological fluid.

41. A system for multiplexed detection of biomarkers on a particle surface comprising:
a plurality of detection elements, wherein the detection elements are configured to detect a passing particle based on an electrical parameter associated with the particle passing the detection element;
a plurality of modulation elements, wherein adjacent detection elements are separated by a modulation element, wherein each modulation element comprises a functionalized surface that is different in composition from a functionalized surface of another modulation element;
a fluid conduit that fluidically connects adjacent detection and modulation elements for providing particles suspended in a fluid to the detection and modulation elements;
an electronic system configured to:
obtain an electrical parameter for each particle that passes each detection element, wherein a modulation element positioned between adjacent detection elements is configured to generate a change in the obtained electrical parameter; and
detect a plurality of biomarkers by comparing the obtained particle parameters from adjacent detection elements separated by one of the modulation elements;
a microfluidic pump for forcing the particles suspended in the fluid through the plurality of detection elements and the plurality of modulation elements.

42. A system for multiplexed detection of biomarkers on a particle surface comprising:
a plurality of detection elements, wherein the detection elements are configured to detect a passing particle based on an electrical parameter associated with the particle passing the detection element;
a plurality of modulation elements, wherein adjacent detection elements are separated by a modulation element, wherein each modulation element comprises a functionalized surface that is different in composition from a functionalized surface of another modulation element;
a fluid conduit that fluidically connects adjacent detection and modulation elements for providing particles suspended in a fluid to the detection and modulation elements;
an electronic system configured to:
obtain an electrical parameter for each particle that passes each detection element;
obtain an aggregate particle parameter from a plurality of particles that passes the detection element, wherein each detection element has a unique aggregate particle parameter;
detect a plurality of biomarkers by comparing the aggregate particle parameters from adjacent detection elements separated by one of the modulation elements;
a microfluidic pump for forcing the particles suspended in the fluid through the plurality of detection elements and the plurality of modulation elements.

43. The system of claim 41 or 42, wherein the conduit has a cross-sectional area selected to facilitate single-file flow of particles over each detection element and each modulation element.

44. The system of claim 43, wherein the conduit has a dimension that is between 1.5 D and 10 D, wherein D is an average particle diameter and flow in the conduit is laminar.

45. The system of claim 44, wherein particles interact with a surface of the modulation element.

46. The system of claim 45, wherein the interaction is an adherence interaction, a rolling interaction, or a free-flow velocity that is not substantially decreased by the functionalized surface.

47. The system of claim 41, wherein the detection element comprises an electrode.

48. The system of claim 41 or 42, wherein the functionalized surface of the modulation element comprises a target molecule specific for a biomarker on the particle surface.

49. The system of claim 41 or 42, wherein the detection and modulation elements are arranged in a series configuration, a parallel configuration, or both a series and a parallel configuration.

50. The system of claim 41 or 42, wherein the detection elements are re-useable and the modulation elements are replaceable.

51. The system of claim 50, where the modulation elements are positioned within a removable cartridge in a point-of-care device.

52. The system of claim 41, wherein the detection element detects a physical property of the particle selected from the group consisting of: an electrical property, a mechanical property; and a magnetic property.

53. The system of claim 42, wherein the detection element detects a physical property of the particle selected from the group consisting of: a mechanical property; and a magnetic property.

54. The system of claim 41 or 42, comprising three or more detection elements and two or more modulation elements.
